(19) United States
(12) Patent Application Publication
(10) Pub. No.: US 2023/0248827 A1
(43) Pub. Date: Aug. 10, 2023

(54) Bisphosphonate-Modified Liposomes Containing Nanoparticles

(71) Applicant: Pharma Development Holding GmbH, Düsseldorf (DE)

(72) Inventor: Wolfgang Greb, Düsseldorf (DE)

(21) Appl. No.: 18/012,237

(22) PCT Filed: Jun. 25, 2021
(86) PCT No.: PCT/DE2021/100537

§ 371 Date: Dec. 22, 2022

(30) Foreign Application Priority Data
Jun. 26, 2020 (DE) 10 2020 116 859.4

(51) Int. Cl. A61K 41/00; A61K 49/00; A61K 9/127; A61K 45/06; A61K 47/54

(52) U.S. Cl. CPC A61K 41/0028; A61K 49/0002; A61K 9/1271; A61K 45/06; A61K 47/548; A61K 47/542; A61K 47/554

(57) ABSTRACT

The invention relates to liposomes containing nanoparticles, wherein the nanoparticles are selected from magnetic, zparamagnetic, superparamagnetic and/or fluorescent and/or functionalized nanoparticles, and the liposomal sleeve contains lipid-derivatized bisphosphonic acid. The liposomes are suitable for preparing a solution for the diagnosis of pathological tissue degeneration or conversion processes on the bone and in the bone marrow, in particular for the treatment and diagnosis of bone tumors and bone metastases and disorders in the bone marrow (proliferative diseases of the blood-producing and lymphoreticular system).

The present invention concerns liposomes containing nanoparticles selected from magnetic, paramagnetic, superparamagnetic and/or fluorescent and/or functionalized nanoparticles, as well as a solution containing these liposomes, and the use of liposomally encapsulated nanoparticles for producing a solution for diagnosis of pathologic tissue degradation or conversion processes on the bone and in the bone marrow, in particular for treatment and diagnosis of bone tumors and bone metastases as well as disorders in the bone marrow (proliferative diseases of the blood-producing and lymphoreticular system).

The treatment of bone tumors and bone metastases and proliferative diseases of the bone marrow is realized in general by an open operation, entailing the risk that not all of the tumor cells can be discovered or removed, or by means of radiation treatment with the known side effects such as radiation damages of the surroundings as well as new cell degeneration. The systemic medication-based chemotherapy is in general also too unspecific, the active ingredients do not reach the site of action easily and show too many side effects. Since bone tumors belong to the rather rare tumors, up to now few cell-specific tumor medications have been developed. The same applies also to bone metastases of other origin because their cell-specific medications do not easily reach this site of action and not in the quantities required for the treatment of bone metastases. However, bone metastases are a very frequent phenomenon: Of 100 humans who die of cancer, 35 have bone metastases, are therefore rather frequent and preferred in case of breast cancer, prostate cancer with 7 of 10 patients in the metastasized disease stage, followed by lung cancer, kidney cell cancer, and thyroid cancer.

In the treatment of different bone diseases and diseases concerning the calcium metabolism, and also in case of diseases as Paget's disease, hypercalcemia, osteoporosis, and neoplasias, bisphosphonates are used frequently.

A further advantage of certain bisphosphonates is that they can cause the apoptosis of tumor cells. Therefore, they play an important role in cancer therapy (e.g. in case of breast cancer, metastases caused by prostate cancer, or in case of multiple myeloma).

Bisphosphonates are pyrophosphate analogues in which the oxygen bridge is replaced by a carbon atom with varying side chains. The P-C-P group is resistant relative to enzymatic hydrolysis; for this reason, bisphosphonates are metabolized badly in the body. Bisphosphonates can be classified into three generations. They differ in the substitution of the hydrogen by different side chains and two possible positions in the molecule. Alkyl side chains (e.g. etidronate) characterize the first generation. The second generation of bisphosphonates comprises the amino-bisphosphonates with a terminal amino group (e.g., alendronate). Side chains that comprise rings are typical for the third generation (e.g. zoledronate).

In bone scintigraphy, phosphonates are used regularly as diagnostic agents. Some differently marked phosphonates, such as e.g. ^{99m}TC-marked phosphonates or ¹⁸⁸Re complexes, are used as radioactive markers in order to show in the skeleton the presence, the location, and the degree of diseases, such as osteomyelitis, bone and bone marrow neoplasias or arthritis.

The most important pharmacological effect of bisphosphonates is the inhibition of bone resorption. They have, like the pyrophosphate, a high affinity to hydroxylapatite, the main component of bone, and prevent its growth as well as its decomposition. In addition, they inactivate bone-decomposing cells, referred to as osteoclasts, in that they cause their apoptosis. Normally, the osteoclasts interact with the bone-building cells, the osteoblasts, in order to rebuild the existing bone. They focus on bone areas which exhibit a high osteoclast activity and they contribute to restoring the normal ratio between osteoblast activity and osteoclast activity.

In the past years, various new therapy forms for focal treatment of tumor diseases have been developed also. For example, tumors can also be destroyed locally by so-called thermal ablation. This concerns a treatment method in which cell tissue, in general tumor tissue, is destroyed by local application of heat, e.g. via wire probes, microwaves, radio waves, or electromagnetic alternating fields. They are employed against various benign and malign tumors, e.g., against liver metastases or against benign thyroid nods and provide a gentle alternative to operation. A special and particularly gentle case is the focal alternating field thermal ablation by means of nanoparticles: after injection of magnetic iron nanoparticles in or at the tumor tissue, they are heated by externally applied electromagnetic alternating fields and the tissue surrounding the nanoparticles is locally destroyed.

WO 2006/108405 discloses nanoparticle-active ingredient conjugates which contain magnetic nanoparticles to which at least one therapeutically active substance is chemically bonded or adsorbed. The detachment of the therapeutically active substance from the nanoparticle is effected or initiated by a magnetic alternating field. A thermally initiated cleavage is possible in which a local heating to above 45° C. —preferably above 50° C. —under bodily conditions is realized.

In particular, cholesteryl-trisoxyethylene-bisphosphonic acid liposomes (CHOL-TOE-BP liposomes) are suitable for a stable and longer residence time in the blood as well as the targeted transport of active ingredients to apatite-containing structures, such as e.g. bone, for treatment of pathological conversion or degradation processes (tumors) as well as for a release of active ingredients at the respective site of action by disintegration/degradation of the liposomes which, in turn, thus releases active ingredients with delay into the bone marrow and into the bloodstream.

In this manner, a preferred enrichment of active ingredients in the bone tissue is achieved, in the bone cells (osteoblasts/osteoclasts/tumor cells) and also in the bone marrow (blood-producing and lymphoreticular organ in the caverns of the bone): In the bone marrow—in particular in the long hollow bones—the blood production and maturation of the various blood cells takes place (red and also white blood cells: erythrocytes, leucocytes, lymphocytes etc.). Here, pathological processes such as pathologic cell formation (tumors such as leukemia) can also take place, and the cell maturation (e.g. of immune cells) can be influenced.

The present invention therefore was based on the object to provide a method which transports diagnostic and/or therapeutic active ingredients in a targeted fashion to the bone and in particular to diseased sites at or in the bone in order to become active thereat in a focused manner. Finding a transport system for nanoparticles with specific physical properties such as paramagnetic behavior (for use in thermal ablation and as NMR marker), intrinsic fluorescence (for visual detection of the site by appropriate light sources (e.g. UV light) with and without specific tumor receptor binding sites as well as for chemical or biological active ingredients and medication-based active ingredients was the goal. One of the prerequisites for the diagnostic and/or therapeutic active ingredients to arrive at the site of action is that the transport system, i.e., the liposomes laden with active ingredients, are sufficiently stable in order to arrive in the target organ/at the site of action.

Subject matter of the present invention are liposomes containing nanoparticles, wherein the nanoparticles are selected from magnetic, paramagnetic, superparamagnetic and/or fluorescent and/or functionalized nanoparticles and the liposomal envelope contains lipid-derivatized bisphosphonic acid.

With these liposomes used according to the invention it is possible to transport nanoparticles used for diagnostics and also for therapy, e.g. the particles by means of a solution, for example, a peripheral infusion solution or local injection solution, in a targeted fashion to the site of action at the bone and to accumulate them specifically in the vicinity of the tumor. In individual cases, liposomal solutions can even be administered by inhalation. The liposomes according to the invention can also be referred to as function liposomes. They can be used as targeted transport systems to the special site of action, e.g. for a local treatment of tumors of the bone or metastases at the bone, e.g. for thermal ablation.

The expression “targeted active ingredient application” means that systems (envelope+enveloped active ingredient or nanoparticle or active ingredient solution with nanoparticle) are used which enable a time-controlled release, an organ-specific application, active ingredient protection, retarded release, and in vivo action, and a decrease of toxicity of the active ingredients. Many carrier systems, such as e.g. polymers, nanoparticles, microspheres, micelles, protein carrier systems, DNA complexes, as well as liposomes, have been used in order to bring active ingredients to the desired site of action, to extend the circulation time of various molecules, and in order to protect them from decomposition in the body/blood/or plasma. Liposomes have been used up to now in various ways as active ingredient carriers. They comprise colloidal, vesicular structures on the basis of (phospho) lipid bilayers. Because of these structural properties, they can incorporate and transport hydrophilic as well as hydrophobic molecules. In addition, the carrier liposomes can be biologically decomposed and are substantially non-toxic because they are comprised of natural biomolecules.

Liposomes have the advantage that the release of the magnetic nanoparticles and of the possibly present active ingredients is delayed. Moreover, these substances are protected against fast decomposition and metabolism and also against diffusion loss. A targeted transport with protection of the contents is in particular important for the medical application of nanoparticles because, in case of a simple injection into the bloodstream or into the tissue, the particles, due to the small dimension and minimal size, immediately diffuse into the surroundings and a spot-on application with residence at the site of action is possible only with difficulty without expedients. Therefore, the use of special transport systems such as liposomes is required for diagnostic and/or therapeutic applications of nanoparticles.

Liposomal formulations are typically used/employed for pharmaceutical “slow release” formulations. Thereby, the pharmaceuticals which are transported in the liposomal vesicles in the blood or directly present in the tissue by local injection remain bioavailable for a longer time and effective for a longer time. Due to the incorporation of cholesterol-bisphosphonate into the liposome envelope, bisphosphonate groups project to the exterior which causes the additional apatite-searching (transport) and local (release) absorption and resorption of the contents of the liposomes. In addition, the transported contents is available at or in the vicinity of the binding site due to the local release from the liposomes.

This transported contents itself can bind to apatite (e.g. bisphosphonate ferrofluids) other active ingredients can bind to/react with the tumor cell receptors (e.g. EGF receptor-active anti-tumor substances to metastases in case of colon cancer), uncoated nanoparticles e.g. for heating/thermal ablation deposited but also quantum-physical “functional” nanoparticles (such as e.g. paramagnetic nanoparticles/ferrofluids or e.g. fluorescent nanoparticles such as QDs or nanoparticles/carriers coated with therapeutic active substances or diagnostics or uncoated as well as unbonded substances in solution can selectively reach the bone as site of action.

A further application possibility is the use of the liposomes according to the invention containing paramagnetic ferrofluids for diagnostic detection in MRT.

Due to the exceptional affinity and binding of the bisphosphonate group to the hydroxylapatite of the bone, its use for the targeted application of pharmacologically active substances to the bone has been examined also. Examples therefore are: radioisotopes, anti-neoplastic active ingredients, and anti-inflammatory substances.

Bisphosphonates which can be used in the context of the present invention are known from the prior art and, for example, are disclosed in WO 2005/070952. Preferably, a lipid-derivatized bisphosphonic acid with the following formula I is employed:

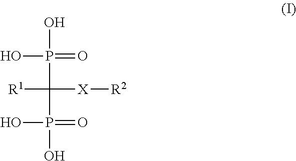

wherein R¹ is H, OH, C₁-C₆-alkyl, C₁-C₆-alkoxy, C₁-C₆-hydroxyalkyl, C₁-C₆-aminoalkyl, C₁-C₆-halogenalkyl is OH,
   X is a direct bond, an alkylene group with 1 to 20 carbon atoms, (CH₃)ₘ—(OCR³HCH₂)ₙ—(O)ₒ—, in which R³ means H or CH₃ and m is 0 or a number from 1 to 6, n is a number from 1 to 10, in particular 1 to 6, and o is 0 or 1, —(CR⁴HCH₂O)ₚ—, R⁴ means H or CH₃, p is a number from 1 to 10, in particular 1 to 6, (CH₃)_{q}—(OCR—⁵HCH₂)ᵣ—(O)ₛ—(CH₃)ₜ—, in which R⁵ means H or CH₃, and q is 0 or a number from 1 bis 6, r is a number from 1 to 10, in particular 1 to 6, and s is 0 or 1, und t is a number from 1 to 6,
   R² is a substituent with the formula (II)

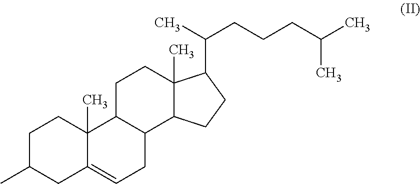 [SMILES: CC(C)CCCC(C)C1CCC2C3CC=C4CC(C)CCC4(C)C3CCC12C]

or a fatty acid chain with 8 to 22 carbon atoms, wherein the substituents with the formula II and the fatty acid chain can comprise substituents such as halogen, in particular F,

as well as their physiologically acceptable derivates, in particular salts and trimethylsilyl derivates.

The bisphosphonic acid compounds employed in the liposomes according to the invention can be present in the form of their acids but also as salts or trimethylsilyl derivatives. In the trimethylsilyl derivatives, at least one of the OH groups at the P is replaced by a trimethylsilyl group. As salts, all physiologically acceptable salts are conceivable, in particular the alkaline, alkaline earth, and ammonium salts.

Particularly preferred are such compounds with the formula I in which R¹ is OH and R² is a substituent with the general formula (II) (i.e., cholesteryl-3-hydroxy-bisphosphonic acid), its soluble salts thereof, with or without spacer molecule. When the substituent R is a fatty alkyl substituent, the latter is preferably selected from fatty alkyl substituents with 12 to 18 carbon atoms, such as a substituent derived from dodecanoic acid or palmitic acid, i.e., the compounds with the formula I are dodeca-bisphosphonic acid or palmityl-bisphosphonic acid.

In a preferred embodiment, the liposomal envelope contains a compound with the general formula I, phospholipids, and/or a uronic acid derivative. The afore described bisphosphonates are characterized by a high affinity to the bone and are suitable as expedient for the active ingredient as well as for the transport of diagnostics, for example, supermagnetic particles, radioactive particles etc. The nanoparticles employed according to the invention comprise the above described bisphosphonic acids as envelope. In a preferred embodiment of the present invention, the envelope represents a liposomal encapsulation. Liposomes comprise colloid, vesicular structures on the basis of (phosphorus) lipid double bilayers. Because of these structural properties, they can incorporate hydrophilic as well as hydrophobic molecules. They are decomposable and substantially non-toxic because they are comprised of natural biomolecules.

The liposomes according to the invention comprise preferably a particle size of 50 nm to 200 μm (0.05 μm to 200 μm), in particular of 100 nm-250 nm. Such a particle size enables a stable transport of the liposomes to the site of action in the bone tissue.

According to the invention, the liposomes contain nanoparticles that are selected from magnetic and/or fluorescent nanoparticles. Suitable nanoparticles should have such a size that they can be encapsulated by liposomes and can comprise a size of 5 to 450 nm. A common particle size of the nanoparticles amounts to 5-20 nm. Preferably, the nanoparticles are selected from nanoparticles of iron oxides, pure iron with an oxide layer, ferrofluids, QDs, gadolinium, silicate, gold or carbon particles coated with magnetic or fluorescent substances, and any mixtures thereof.

The magnetic particles that are contained in the nanoparticles employed according to the invention are magnetic particles known from the prior art. They are comprised of a magnetic material, preferably a ferromagnetic, anti-ferromagnetic, ferrimagnetic, anti-ferrimagnetic or superparamagnetic material, further preferred of iron oxides, in particular superparamagnetic iron oxides or of pure iron which is provided with an oxide layer. The scope of the present invention encompasses also paramagnetically coated QDs (quantum dots), QDs which contain an Fe core or silicate-coated nanoparticles with a magnetic core as well as fluorescent and also radioactive substances or solutions, such as solutions of Tc-99, C-14, and stable isotopes, such as C-13, F-19, which can be used in MRT in bisphosphonate liposomes. The aforementioned particles can be heat-activated by means of electrical alternating fields.

In a possible embodiment, the liposomes according to the invention are heated by a magnetic alternating field. Heating of the tissue containing the nanoparticles to more than 50° C. is possible. Such high temperatures can be reached because up to 800 pg and more of iron in the form of the nanoparticles can be taken up per tumor cell.

Preferably, the nanoparticles are comprised of iron oxides and in particular of magnetite (Fe₃O₄), maghemite (γ-Fe₂O₃), or mixtures of these two oxides. In general, the preferred nanoparticles can be represented by the formula FeOx wherein x is a number from 1 to 2. The nanoparticles comprise preferably a diameter of less than 500 nm. Preferably, the nanoparticles have an average diameter of 15 nm or lie preferably in the size range of 1 to 100 nm and particularly preferred in the range of 10 to 20 nm.

In addition to the magnetic materials of the formula FeOx wherein X is a number in the range from 1.0 to 2.0, according to the invention also materials of the general formula MFe₂O₄ with M═Co, Ni, Mn, Zn, Cd, Ba, Gd or other ferrites can be used. Moreover, also silica-carbon or polymer particles are suitable in which the magnetic materials such as, for example, the herein mentioned magnetic materials, are incorporated and/or bonded.

Preferably, these particles are comprised of magnetic iron oxides or of pure iron with an oxide layer. These magnetic particles can be, for example, produced according to the method disclosed in DE 4428851.

In a further possible embodiment, the liposomes contain fluorescent nanoparticles, such as, for example, particles of silica or calcium phosphate doped with a dye or surface-modified semiconductor particles, such as those of binary compounds such as lead sulfide, lead selenide, cadmium selenide, cadmium sulfide or cadmium telluride or of ternary compounds such as cadmium selenide sulfide, zinc selenide, which in the biological research as imaging agents or in clinics as local markers or reference points for detection of NP by means of X-ray, carbon nanoparticles (Indian ink) and gold nanoparticles. Particularly suitable particles are also so-called QDs (quantum dots) which are superior to known fluorescent dyes due to their intensive fluorescence and photostability. QDs on the basis of zinc selenide are particularly preferred because of their fluorescent properties, good functional properties, and minimal toxicity. The excitation of the fluorescent nanoparticles is realized in conventional manner known to a person of skill in the art, such as photo excitation by means of suitable light sources.

In a further embodiment, the nanoparticles are selected from carbon nanoparticles which can also be coated and/or functionalized and can be imparted with semiconductor properties by the functionalization. The carbon nanoparticles have the advantage that they are less toxic in comparison to iron-containing QDs.

The nanoparticles and in particular the QDs can be bonded to proteins, oligonucleotides, smaller molecules etc. in order to bind them immediately to the target at the bone.

In a possible embodiment, the nanoparticles comprise a protective envelope or functionalized coating.

This protective envelope or coating can also comprise a functionalization of the surface. The functionalization of the surface comprises free amino groups, hydroxide groups, carboxyl groups or carbonyl groups to which an active ingredient or a functional linker can be bonded by means of an imine bond, amine bond, ester bond, amide bond or ketal bond. By means of this linker, also a therapeutically active or diagnostic substance—e.g. a receptor-binding antibody—can be bonded covalently, ionically, by complexing, lipophilically or by hydrogen bonds. The production of particles with a protective envelope and optionally a functionalization can be realized according to methods as they are disclosed in WO 2006108405.

In addition to the magnetic nanoparticles, the liposomes according to the invention can also contain therapeutically active and/or diagnostically active substances. These substances are transported by means of the liposomes directly to the site of action and can be released thereat. The release is realized usually spontaneously when the liposomal particle has reached the site of action/target, decomposes or the thermal ablation is carried out.

In a further embodiment, the optionally contained therapeutically active substances are not directly bonded to the magnetic particles but can be present within the envelope which contains the lipid-derivatized phosphonic acid. In case of a liposomal encapsulated form, the therapeutic as well as diagnostic active ingredient as well as the magnetic particles are liposomally encapsulated. The active ingredient and the magnetic particles can be present together encapsulated in a liposome but also in separate liposomes.

These substances can be present within the liposomal envelope or can be bonded to the envelope. For example, they can be located at the surface of the nanoparticles and/or present at the nanoparticles without being bonded thereto. At the surface, the substances can be bonded by binding locations, for example. Bonding to the surface can be covalent such as by a functional group arranged at the surface, any other bonding such as an ionic bond or other interactions. In a preferred embodiment, the diagnostically or therapeutically active substances are bonded to the lipid-derivatized bisphosphonic acid, for example, by a covalent bond, an ionic bond and/or by van der Waals interaction.

The therapeutically active substances can be selected from chemical or biological therapeutically active substances such as antiproliferative, antimigratory, antiangiogenic, antithrombotic, anti-inflammatory, antiphlogistic, cytostatic, cytotoxic, immunotherapeutic, anticoagulative, antibacterial, antiviral and/or antimycotic substances as well as vaccines. Particular preferred are antiproliferative, antimigratory, antiangiogenic, cytostatic and/or cytotoxic substances as well as nucleic acids, amino acids, peptides, proteins, carbohydrates, lipids, glycoproteins, glycans or lipoproteins with antiproliferative, antimigratory, antiangiogenic, antithrombotic, anti-inflammatory, antiphlogistic, cytostatic, cytotoxic, anticoagulative antibacterial, antiviral and/or antimycotic properties.

As cytotoxic and/or cytostatic components, for example, alkylation agents, antibiotics with cytostatic properties, anti-metabolites, microtubuli inhibitors and topoisomerase inhibitors, platinum-containing compounds, and other cytostatic agents, such as, for example, asparaginase, tretinoin, alkaloids, podophyllotoxins, taxanes, and Miltefosin®, hormones, immunomodulators, monoclonal antibodies, signal transducing agents (signal transduction molecules), and cytokines can be used.

As diagnostic substances, all conventional diagnostic agents which are used in clinical day-to-day operation and specialized centers can be used which are suitable in radiological methods such as CT, X-ray, MRT, NMR, and in the nuclear medical, such as isotope scintigraphy/Gamm camera, positron emission tomography (PET) and as radiopharmaceuticals. These substances also include therapeutically and/or diagnostically active substances such as contrast agents for imaging methods, radionucleotides, antibodies, and tumor markers.

Tumor markers are biochemical substances which, for some cancer types, are produced by the tumor cells, are expressed/exist on their cell surface, and are released into the blood. Accordingly, they can be diagnostically detected with sensitive methods on the tumor cells or in the blood of the patient.

Tumor markers are often built of sugars and proteins (so-called glycoproteins) such as e.g. the carcinoembryonic antigen (for short CEA), a marker for colon cancer. In addition to glycoproteins, hormones, and enzymes, genetic diagnostics are increasingly used. When a tumor shows certain genes (gene expression), this can be an indication of the special tumor cell type of which a primary tumor or its metastases are comprised.

Correspondingly, one can diagnose, localize and characterize the tumor, and then therapeutically attack it, e.g. operate or destroy in a targeted fashion, e.g. by thermal ablation or, newly, with antibodies as examples of an on-target therapy (“targeted therapy”). In this context, it is very important that as many as possible of the tumor cells are completely removed. This is often more likely the case for an on-targeted attack on the molecular level.

However, not all medications even for intravenous application reach their site of action in a sufficient quantity. Therefore, they must be e.g. locally injected or enriched in the tissue, but injection is hardly possible in case of bone.

For different cancer diseases, there are different markers. The known tumor markers include for example:

and other markers are continuously newly established.

| Tumor Type | Marker |
|---|---|
| Breast cancer | CA15-3, CEA, CA 125, HER2-new |
| Ovarian cancer | CA 125, beta-HCG, AFP |
| Lung cancer | NSE, CYFRA 21-1, SCC |
| Stomach cancer | CEA, CA-72-4, CA 19-9 |
| Colon cancer | CEA, EGFR |
| Pancreatic cancer | Cd 44 |
| Prostate cancer | PSA, PSMA, CG-1 |
| Bone cancer | RANKL |

In a particularly preferred embodiment, the liposomes contain tumor markers.

Various cancer types, such as e.g. breast cancer or prostate cancer, metastasize into the bone. For example, a liposome according to the invention can contain a HER2 antibody-coated nanoparticle, such as e.g. an Fe nanoparticle, and can be injected intravenously as diagnostic agent and, for example, detected by means of MRT.

When as a result of the liposome transport to the bone after spontaneous release in the MRT a positive enrichment of the antibody-laden nanoparticles at the bone is found, these HER2 antibody nanoparticles can be used for therapy of the bone metastasis for a local thermal ablation, but the free antibodies (without Fe particles) can also be transported, liposomally encapsulated, in a targeted fashion to the bone metastases. This would be a treatment in the form of a theranostic system.

The production of the liposomes according to the invention can be carried out with methods known in the prior art. A possible method is, for example, the lipid film extrusion method. The production of liposomes is, for example, disclosed in the dissertation of Verena Hengst (Department of Pharmacy of the Philipps University of Marburg, 2007). Further production methods can be found at: https://de.wikipedia.org/wiki/Liposomenerzeugung.

The liposomes according to the invention are present in a conventional liposomal formulation, for example, in form of a liposomal dispersion. This liposomal formulation can be administered as such, or it can be further processed to a solution (application solution) as is generally known in the art.

As further components, the liposomes and liposome formulations according to the invention can contain expedients known in the prior art for the production of liposomes, such as solvents, rheological agents (dextrans, heparin derivatives), antioxidants, esterase inhibitors, pH buffering substances. In particular pH buffering substances are suitable to influence the stability of the liposomes and their interaction with the target cells.

A further subject matter of the invention is a solution which contains the above-described liposomes and for production of a therapeutic agent, diagnostic agent or a combined theranostic system for diagnosis and/or treatment of pathological tissue degradation or conversion processes at the bone or in the bone marrow, in particular for treatment and diagnosis of bone tumors and bone metastases as well as disorders in the bone marrow (proliferative diseases of the blood-producing and lymphoreticular system).

For example, for diagnosis and/or treatment of bone tumors and bone metastases, own and foreign metastases, and of pathological bone tissue processes. In a possible embodiment, the solution is an infusion solution or an injection solution.

A further subject matter is the use of liposomally encapsulated nanoparticles for producing a solution for focal treatment and/or diagnosis of bone conversion processes, wherein the nanoparticles are selected from magnetic, paramagnetic, superparamagnetic or/and fluorescent and/or functionalized nanoparticles and the liposomal envelope contains lipid-derivatized bisphosphonic acid or bisphosphonic acid derivatives.

Yet another subject matter is the use of the afore described liposomally encapsulated nanoparticles for producing a solution for localization, diagnosis and/or therapy of bone conversion processes.

The solutions (application solutions) are aqueous solutions that comprise a pH value in the physiological range, preferably between 6.8 and 8.0. These solutions, for example, can comprise emulsifiers and stabilizers, buffer systems such as HEPES, and further components that do not impair the stability of the liposomes and enhance the absorption into the cell. For example, the liposomes can be stable but the absorption into the cells is disturbed when the medium of the liposome formulation is not neutral or is too acidic. Also, the charge of the liposome envelope has an influence on the absorption into the cell; it should be as neutral as possible or only weakly negative/acidic.

In a possible embodiment, the liposomes according to the invention are used for producing a diagnostic agent for recognizing, marking and/or agent for removal of tumor lesions (solid tumors and metastases) at or in the bone.

In particular, the liposomes according to the invention are suitable for thermal ablation of tumors and metastases and foreign metastases, in particular in the bone tissue.

The solutions, for example, infusion solutions but also injection solutions, are preferably a physiological saline solution that is suitable for interstitial or intra-tumoral application.

What is claimed is:

1.-14. (canceled)

15. A liposome comprising:
nanoparticles, the nanoparticles selected from magnetic, paramagnetic, superparamagnetic and/or fluorescent and/or functionalized nanoparticles;
a liposomal envelope comprising lipid-derivatized bisphosphonic acid.

16. The liposome according to claim 15, wherein the lipid-derivatized bisphosphonic acid is selected from compounds having the general formula I 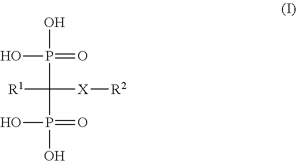
wherein
R¹ is H, OH, C₁-C₆-alkyl, C₁-C₆-alkoxy, C₁-C₆-hydroxyalkyl, C₁-C₆-aminoalkyl, C₁-C₆-halogenalkyl,
X is a direct bond, an alkylene group with 1 to 20 carbon atoms, (CH₂)ₘ—(OCR³HCH₂)ₙ—(O)ₒ—, in which R³ means H or CH₃ and m is 0 or a number from 1 to 6, n is a number from 1 to 10, in particular 1 to 6, and o is 0 or 1, wherein m, n, and o are not 0 at the same time,
—(CR⁴HCH₂O)ₚ—, R⁴ means H or CH₃, p is a number from 1 to 10, in particular 1 to 6,
(CH₂)_{q}—(OCR⁵HCH₂)ᵣ—(O)ₛ—(CH₂)ₜ—, in which R⁵ means H or CH₃, and q is 0 or a number from 1 bis 6, r is a number from 1 to 10, in particular 1 to 6, and s is 0 or 1, und t is a number from 1 to 6,
R² is a substituent with the formula (II) 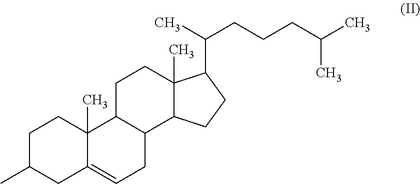 [SMILES: CC(C)CCCC(C)C1CCC2C3CC=C4CC(C)CCC4(C)C3CCC12C]
or a fatty acid chain with 8 to 22 carbon atoms, wherein the substituents with the formula II and the fatty acid chain can comprise substituents such as halogen, in particular F,
and physiologically acceptable derivates thereof, including salts and trimethylsilyl derivates.

17. The liposome as claimed in claim 16, wherein the liposomal envelope further contains phospholipids and/or a uronic acid derivative.

18. The liposome as claimed in claim 15, wherein the nanoparticles are selected from the group consisting of iron oxides, pure iron with an oxide layer, ferrofluids, QDs, gadolinium, silicate particles coated with paramagnetic or fluorescent substances, gold particles coated with paramagnetic or fluorescent substances, carbon particles coated with paramagnetic or fluorescent substances, and mixtures thereof.

19. The liposome as claimed in claim 18, wherein the nanoparticles comprise a protective envelope or a functionalized coating.

20. The liposome as claimed in claim 15, further comprising one or more therapeutically and/or diagnostically active substances.

21. The liposome as claimed in claim 20, wherein the one or more therapeutically and/or diagnostically active substances are present within the liposomal envelope or are bonded to the liposomal envelope.

22. The liposome as claimed in claim 20, wherein the one or more therapeutically active substances are selected from the group consisting of antiproliferative substances, antimigratory substances, antiangiogenic substances, antithrombotic substances, anti-inflammatory substances, antiphlogistic substances, cytostatic substances, cytotoxic substances, immunotherapeutic substances, anticoagulative substances, antibacterial substances, antiviral substances, antimycotic substances, and vaccines.

23. The liposome as claimed in claim 20, wherein the one or more diagnostically active substances are selected from the group consisting of contrast agents for imaging methods, radial nucleotides, and tumor markers.

24. A solution containing liposomes according to claim 15 for producing a therapeutic agent, a diagnostic agent or a combined theranostic system for diagnosis and/or treatment of pathological tissue degradation or conversion processes on the bone or in the bone marrow, including treatment and diagnosis of bone tumors and bone metastases and disorders in the bone marrow.

25. A method of producing a solution for localization and diagnosis of bone conversion processes, the method comprising using a liposome according to claim 15 in the solution.

26. A method of producing a solution for focal treatment of bone tumors and bone metastases, own and foreign metastases in bone tissue, and diseases of the blood-producing and lymphoproliferative system by thermal ablation, the method comprising using a liposome according to claim in the solution.

27. A method for therapeutic thermal ablation of tumors and metastases, the method comprising using the solution according to claim 24.

28. A diagnostic method for recognizing and marking tumor lesions at or in the bone, the method comprising using the solution according to claim 24.
